(19) United States
(12) Patent Application Publication
(10) Pub. No.: US 2009/0304674 A1
(43) Pub. Date: Dec. 10, 2009

(54) METHODS FOR TREATING DISEASE BY REGULATING CLL CELL SURVIVAL

(76) Inventors: Thomas J. Kipps, San Diego, CA (US); Tomoyuki Endo, Hokkaido (JP); Mitsufumi Nishio, Hokkaido (JP)

Correspondence Address: DLA PIPER LLP (US), 4365 EXECUTIVE DRIVE, SUITE 1100, SAN DIEGO, CA, 92121-2133, US

(21) Appl. No.: 11/919,013

(22) PCT Filed: Apr. 24, 2006
(86) PCT No.: PCT/US06/15572

§ 371 Date: Jan. 7, 2009

Related U.S. Application Data
(60) Provisional application No. 60/674,239, filed on Apr. 22, 2005.

(51) Int. Cl. A61K 39/395; C12N 5/00; A61P 35/02; A61K 38/02; C12Q 1/02

(52) U.S. Cl. 424/130.1; 435/375; 514/2; 435/29

(57) ABSTRACT

The present teachings include methods for regulating apoptosis in a cell comprising contacting the cell with an agent capable of neutralizing BAFF or APRIL. In yet another teaching a method for treating leukemia is provided. In yet another embodiment, a method for detecting inhibitors of CLL is provided. These and other features, aspects and advantages of the present teachings will become better understood with reference to the following description, examples and appended claims.

### CROSS-REFERENCE TO RELATED APPLICATIONS

This application claims priority from U.S. Provisional Application Ser. No. 60/674,239 filed on Apr. 22, 2005, which is incorporated herein by reference in its entirety.

### STATEMENT REGARDING FEDERALLY SPONSORED RESEARCH OR DEVELOPMENT

This invention was made in part with Government support under National Institutes of Health Grant CA081534. The Government has certain rights in the invention.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### FIELD

The present teachings relate to methods for treating diseases by regulating Chronic Lymphocityc Leukemia (“CLL”) cell survival.

### INTRODUCTION

Existing therapies for CLL include chemotherapies such as the administration of fludarabine, chlorambucil and the like to patients suffering from CLL. Another therapy is antibody therapy such as administering rituximab to a CLL patient. However, such therapies have substantial side effects such as damage caused to not only malignant cells but also to normal tissue. Therefore, what is needed is a therapeutic strategy based not on killing cancerous cells directly, as is contemplated with the above chemotherapies and antibody therapies, but to interrupt a cancerous cell survival factor from supporting cells. Such a therapy would be less harmful to normal tissue than existing therapies.

### SUMMARY

The present teachings include methods for regulating apoptosis in a cell comprising contacting the cell with an agent capable of neutralizing BAFF or APRIL. In yet another teaching a method for treating leukemia is provided. In yet another embodiment, a method for detecting inhibitors of CLL is provided. These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1.

FIG. 2.

FIG. 3.

FIG. 4.

FIG. 5.

FIG. 6.

FIG. 7.

FIG. 8.

FIG. 9.

FIG. 10.

FIG. 11.

FIG. 12.

FIG. 13.

FIG. 14.

FIG. 15.

FIG. 16.

FIG. 17.

### DETAILED DESCRIPTION

### Methods for Treating Disease by Regulating CLL Cell Survival

We examined expression of B cell-activating factor of the TNF family (BAFF) and a proliferation-inducing ligand (APRIL) on chronic lymphocytic leukemia (CLL) B cells and nurselike cells (NLC), which differentiate from CD14+ cells when cultured with CLL B cells. NLC expressed significantly higher levels of APRIL than monocytes and significantly higher levels of BAFF and APRIL than CLL B cells. Also, the viability of CLL B cells cultured with NLC was significantly reduced when CLL B cells were cultured with decoy receptor of B-cell maturation antigen (BCMA), which can bind both BAFF and APRIL, but not with BAFF-R:Fc, which only binds to BAFF. The effect(s) of BAFF or APRIL on leukemia cell survival appeared additive and distinct from that of stromal cell-derived factor-1 alpha (SDF-1αα), which in contrast to BAFF or APRIL induced leukemia-cell phosphorylation of p44/42 mitogen-activated protein-kinase (ERK1/2) and AKT Conversely, BAFF and APRIL, but not SDF-1α, induced CLL-cell activation of the NF-kappa B1, and enhanced CLL-cell expression of the anti-apoptotic protein Mcl-1. However, BAFF, but not APRIL, also induced CLL-cell activation of NF-kappa B2. We conclude that BAFF and APRIL from NLC can function in a paracrine manner to support leukemia cell survival via mechanisms that are distinct from those of SDF-1α, indicating that NLC use multiple distinct pathways to support CLL-cell survival. BAFF is tumor necrosis factor ligand superfamily, member 13b (285 amino acid). Reference number in NCBI is NP-006564. APRIL is tumor necrosis factor ligand superfamily, member 13 (250 amino acid). Reference number in NCBI is NP-003799.

B-cell chronic lymphocytic leukemia (CLL) is characterized by the accumulation of monoclonal B-cells in the blood, secondary lymphoid tissues, and marrow. The leukemia cells primarily are arrested in the G0/G1-phase of the cell cycle and appear resistant to programmed cell death. Despite their apparent longevity in vivo, CLL cells typically undergo spontaneous apoptosis under conditions that support the growth of human B cell lines in vitro. This implies that the factors essential for survival are not intrinsic to the CLL B cell.

In vitro a subset of blood mononuclear cells from patients with CLL can differentiate into large, round, adherent cells that can attract leukemia cells and protect them from undergoing apoptosis.9 When removed from these cells, the CLL B cells experience a rapid decline in viability. Because these cells attract CLL B cells, share features in common with thymic nurse cells, and support CLL B cell survival, the adherent cells are termed nurselike cells, or NLC.

Subsequent studies found that NLC differentiated from CD14+ blood mononuclear cells upon co-culture with leukemia cells in vitro. Nevertheless, despite expressing myelomonocytic antigens, NLC were found to have an expression profile of surface and cytoplasmic antigens (CD14low, CD68high, CD83negative, CD106negative) that is distinct from those of monocytes, macrophages, or blood-derived dendritic cells. Abundant cells with the morphology and phenotype of NLC are present in secondary lymphoid issues of patients with CLL, suggesting they might also function to promote leukemia cell survival in vivo.

The mechanisms whereby NLC promote CLL cell survival are not resolved. NLC express high-levels of stromal-derived factor-1 alpha (SDF-1α), a CXC chemokine capable of inducing chemotaxis, phosphorylation of mitogen activated protein kinases (MAPK), and improved survival of CLL cells in vitro. Nevertheless, the viability of CLL B cells cultured with even high concentrations of SDF-1α is not as high as that achieved by co-culture with NLC, indicating that factors other than SDF-1α also might be responsible for promoting CLL B cells survival by NLC in vitro.

Investigators have reported that CLL cells express B-lymphocyte stimulator (BLyS), otherwise known as B cell-activating factor of the tumor necrosis factor family. BAFF is a type II transmembrane protein that can act in a membrane-bound or soluble form to promote B cell survival (reviewed by Mackay and colleagues). Moreover, in mice, disruptive mutations of either BAFF or its receptor, BAFF-R, causes profound loss of mature B cells, indicating that BAFF-BAFF-R interactions are critical for the differentiation and/or survival of mature B cells. CLL B cells also were found to express the primary BAFF receptor (BAFF-R), as well as two other receptors that can interact with BAFF, namely B-cell maturation antigen (BCMA) and transmembrane activator and calcium modulator and cyclophilin ligand interactor (TACI). Kern and colleagues also detected expression of BAFF on the surface of CLL cells, implying that BAFF may function in an autocrine manner to support CLL B cell survival.

Two of the BAFF receptors, namely BCMA and TACI, also can bind a proliferation inducing ligand (APRIL), a factor that also can contribute to B cell survival. The third receptor for BAFF, namely BAFF-R, is specific for BAFF and cannot bind to APRIL. APRIL originally was found in tumor cells and supposedly is expressed primarily as a secreted soluble molecule through the action of furin proteases present in the Golgi. However, Kern and colleagues reported that CLL cells also can express surface APRIL, and suggested that this factor also may function as a autocrine survival factor in this disease.

Whether the expression of BAFF and/or APRIL on CLL cells is sufficient for optimal leukemia cell survival is not known. Of note, addition of recombinant BAFF could significantly enhance leukemia cell viability, suggesting that the amount of BAFF expressed on isolated CLL cells may be insufficient to support leukemia cell survival, at least in vitro. Because of the noted dependency of leukemia B cells on accessory cells such as NLC for survival in vitro, and presumably in vivo, we examined the blood mononuclear cells, NLC, and isolated leukemia cells of patients with CLL for their relative expression of BAFF and APRIL.

Expression of BAFF mRNA and Protein on CLL Cells and NLC

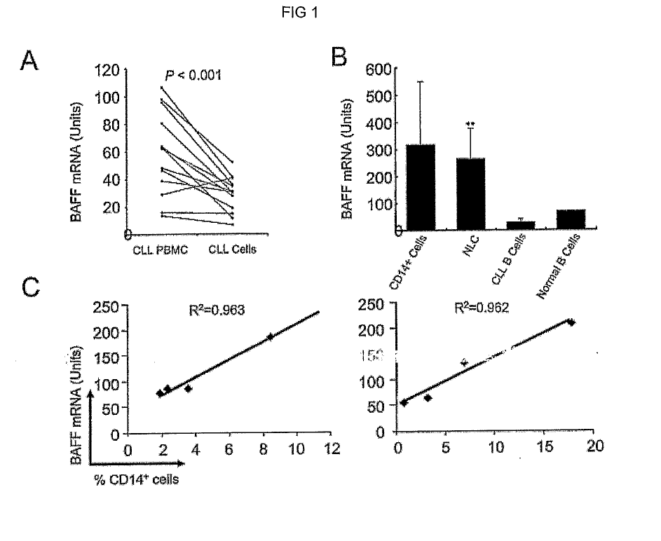

We examined the peripheral blood mononuclear cells (PBMC) of patients with CLL for expression of BAFF mRNA by real-time RT-PCR. In each case, we detected expression of BAFF mRNA, consistent with earlier reports. Moreover, we found that rigorous depletion of CD14+ cells from the PBMC significantly lowered the amount of BAFF mRNA detected in each sample (59±30 Units in PBMC and 29±13 Units in isolated CLL B cells, n=12, P<0.001, paired t test, FIG. 1A). Furthermore, the amount of BAFF mRNA detected in CD14+ cells (320±230, n=4) or NLC (270±110, n=12) was significantly greater than that noted in the isolated leukemia B cells (P<0.0001, FIG. 1B) or isolated CD19+ blood B cells of healthy donors.

Small numbers of CD14+ cells present in the blood mononuclear cells isolated from patients with CLL potentially could contribute a large proportion of the BAFF mRNA detected by real-time RT-PCR assay, which uses GAPDH mRNA to normalize the assay. To evaluate this possibility we added small numbers of CD14+ blood mononuclear cells to purified CD19+ CLL B cells and examined how this affected the amount of BAFF mRNA detected in each sample (FIG. 1C). For each 1% of added CD14+ cells there was an increase in the detected amount of BAFF mRNA of 10-13 Units. At the y-intercept of each graph (FIG. 1C) when the proportion of CD14+ cells was extrapolated to 0%, we detected 30-40 Units of BAFF mRNA. We attribute this to the amount of BAFF mRNA expressed by CLL B cells themselves, as this is the amount we detected in the isolated leukemia B cells (FIG. 18; e.g. 29±13 Units). This implies that on a cell-per-cell basis, CD14+ cells apparently contain approximately 30-fold more BAFF mRNA than CLL B cells.

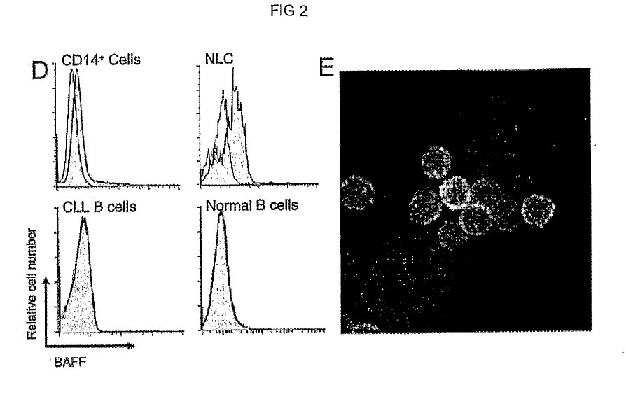

We next examined CLL cells and NLC for surface expression of BAFF by flow cytometry. In contrast to CLL B cells or purified normal B cells, NLC expressed high-levels of BAFF that were easily detected by flow cytometry (FIG. 2D), or immunofluorescence microscopy (FIG. 2E). These data indicate that NLC express large amounts of BAFF protein relative to that expressed by CLL B cells.

FIG. 1(A) provides the results of quantitative real-time RT-PCR was performed on RNA samples isolated from the blood mononuclear cells of individual patients with CLL before (left) and after (right) depletion of CD2+ and CD14+ cells. The lines connect the pre- and post-isolation levels of BAFF mRNA detected in each sample. The amount of BAFF mRNA detected is indicated in arbitrary units. The amount of BAFF mRNA detected in an equivalent number of U937 cells is 1,000 Units (data not shown).

FIG. 1(B) provides the results of quantitative realtime RT-PCR measurement of the average amount of BAFF mRNA detected in CD14+ cells (n=4), NLC (n=12), purified CLL B cells (n=12) and isolated CD19+ blood B cells of normal donors (n=2), as indicated at the bottom of the panel (** indicates that the level of BAFF mRNA detected in NLC was significantly greater than that found in isolated CLL B cells, P<0.0001).

FIG. 1(C) provides the results of reconstitution experiments in which small numbers of CD14+ blood mononuclear cells are added to 5×106 isolated CLL B cells that subsequently were evaluated for BAFF mRNA in two representative patients. On the x-axis is the percent of CD14+ cells detected by FACS in the reconstituted cell population prior to extraction of RNA. The y-axis indicates the level of BAFF mRNA detected in Units. FIG. 2(D) Representative histograms depicting surface BAFF detected by flow cytometry on CD14+ cells, NLC, CD19+ CLL B cells, or CD19+ blood B cells of healthy donors, as indicated at the top of each graph. Shaded histograms represent the fluorescence of cells stained with a fluorochrome-labeled ant-BAFF mAb, whereas the clear histograms depict the fluorescence of cells stained with an isotype control mAb. FIG. 2(E) An immunofluorescence picture of an NLC and CLL cells stained with fluorescein-labeled anti-CD19 mAb (green) and a phycoerythrin-labeled anti-BAFF mAb (red). The nuclei are labeled blue with Hoechst 33342.

Expression of APRIL mRNA and Protein on CLL Cells and NLC

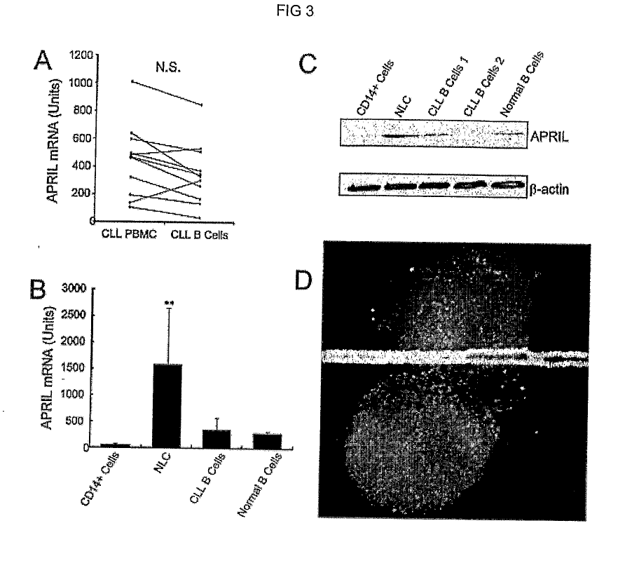

We also examined the PBMC of patients with CLL for expression of APRIL mRNA with the same techniques used for evaluating the expression of BAFF. In contrast to our studies on BAFF mRNA, we found that rigorous depletion of CD14+ cells from the PBMC did not lower the amount of APRIL mRNA detected in each sample tested (440±308 Units in PBMC and 348±228 Units in isolated CLL B cells, n=11, NS, paired t test, FIG. 3A). This indicates that CD14+ blood mononuclear cells do not contribute significantly to the amounts of APRIL mRNA found in CLL blood mononuclear cells. Consistent with this, we found that isolated CD14+ cells had very low amounts of APRIL mRNA (52±20, n=5).

In contrast, the amounts of APRIL mRNA detected in differentiated NLC was significantly higher (FIG. 3B; 1595±1090, n=11) than that of non-differentiated CD14+ blood mononuclear cells. Moreover, NLC had significantly greater amount of APRIL mRNA than that noted in the isolated leukemia B cells or isolated CD19+ blood B cells of normal donors (P<0.01, Bonferroni t test, FIG. 3B).

We evaluated for expression of APRIL by immunoblot analysis. As seen in FIG. 3C, total lysates from NLC had higher amounts of APRIL than did CD14+ blood mononuclear cells, purified CLL B cells, or isolated CD19+ blood B cells of normal donors. NLC also were found to express high levels of APRIL relative to CLL B cells by immunofluorescence staining (FIG. 3D).

FIG. 3(A) provides the results of quantitative real-time RT-PCR was performed on RNA samples isolated from the blood mononuclear cells of patients with CLL before (left) and after (right) depletion of CD2+ and CD14+ cells. The lines connect the pre- and post-isolation levels of APRIL mRNA in each sample. The amount of APRIL mRNA detected is indicated n arbitrary units. The amount of APRIL mRNA detected in an equivalent number of U937 cells is 30 Units (data not shown). FIG. 3(B) Quantitative real-time RT-PCR measurement of the average amount of APRIL mRNA detected in CD14+ cells (n=4), NLC (n=11), purified CLL B cells (n=11), or isolated CD19+ blood B cells of healthy donors (n=3), as indicated at the bottom of the histogram (** indicates that the mean level of APRIL mRNA detected in NLC was significantly greater than that found in isolated CLL B cells, P<0.01). FIG. 3(C) Representative immunoblot data showing the expression of APRIL by NLC, CD14+ blood mononuclear cells, CLL B cells, or isolated CD19+ blood B cells of healthy donors. Whole cell lysates were prepared as described in the Material and Methods section. The protein content was normalized to 20 μg and subjected to immunoblot analysis with antibodies specific for APRIL or β-actin, using ECL-based detection. FIG. 3(D) An immunofluorescence picture of NLC and CLL cells stained with phycoerythrin-labeled anti-CD19 mAb (red) and goat IgG anti-APRIL polyclonal antibody that was detected using a fluorescein-labeled ant-goat IgG (green). The nuclei are labeled blue with Hoechst 33342.

Effect of BCMA-Fc or BAFF-R:Fc on the Viability of CLL Cells Cultured with NLC

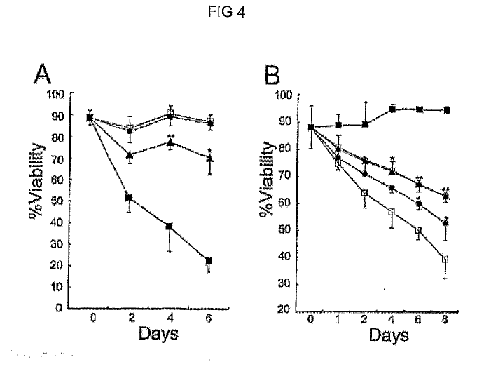

Because NLC express both BAFF and APRIL, we examined whether these factors contributed to the capacity NLC to sustain CLL cell survival in vitro. We cultured CLL B cells with decoy receptors of BCMA (BCMA-Fc), which can bind to both BAFF and APRIL, and BAFF-R (BAFF-R:Fc), which binds to only BAFF, and compared the viability of the leukemia cells with that of such cells cultured with control immunoglobulin (control Ig). We observed that addition of BCMA-FC to co-cultures of CLL cells and NLC significantly reduced the viability of the CLL cells relative to that of co-cultures treated with control Ig (FIG. 4A). In contrast, there was no decline in leukemia-cell viability in such co-cultures when we added saturating amounts of BAFF-R:Fc (FIG. 4A), which in parallel studies were found capable of inhibiting B cell survival in co-cultures with rhBAFF or fibroblast-like synoviocytes that expressed BAFF, but not APRIL (data not shown).

FIG. 4(A) shows the inhibition of CLL-cell survival on NLC by BCMA-Fc, but not BAFF-R:Fc CLL B cells were cultured with (open squares) or without (closed squares) NLC and 1 μg/ml control Ig. BCMA-Fc (closed triangles) or BAFF-R:Fc (closed circles) at 1 μg/ml was added to the wells of CLL B cells cultured with NLC at day 0. Viability was subsequently determined for each time point, as indicated on the horizontal axis. Displayed are the mean percent viability ±S.D. (error bars) of samples from each 5 patients. The percent viability of BCMA-Fc treated cultures was significantly less than that of control Ig treated cultures (* indicates P<0.05; ** indicates P<0.01; Bonferroni t test). FIG. 4(B) Enhanced CLL cell survival with NLC or rhBAFF or rhAPRIL 1×106 ml of isolated CD19+ CLL B cells were cultured alone (open squares), with 50 ng/ml rhBAFF (closed triangles), 500 ng/ml rhAPRIL (closed circles), both rhBAFF and rhAPRIL (open circles) or with NLC (closed squares) and evaluated over time. Displayed are the mean percent viability ±S.D. of samples from each 3 patients. The percent viability of rhBAFF-treated CLL cells or rhAPRIL treated CLL cells was significant greater than that of control treated CLL cells (* indicates P<0.05; ** indicates P<0.01; Bonferroni t test).

Additive Effects of SDF-1α and BAFF or APRIL on CLL B-Cell Survival

Next we examined whether NLC or exogenous BAFF or APRIL could enhance the viability of CLL B cells in vitro. For this, we monitored the viability of CLL B cells over time when cultured with or without NLC or with or without rhBAFF or rhAPRIL. Consistent with prior studies, CLL cells cultured alone had less viability than leukemia cells cultured with NLC. The addition of rhBAFF or rhAPRIL significantly improved the viability of CLL cells cultured without NLC (FIG. 4B). The viability of the CLL cells co-cultured with either rhBAFF or rhAPRIL alone was not enhanced further by the addition of rhAPRIL or rhBAFF, respectively.

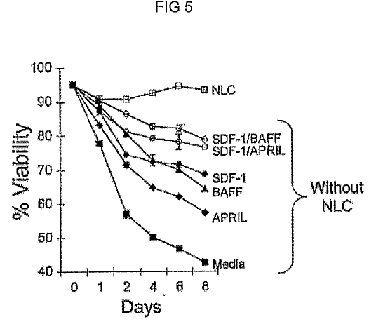

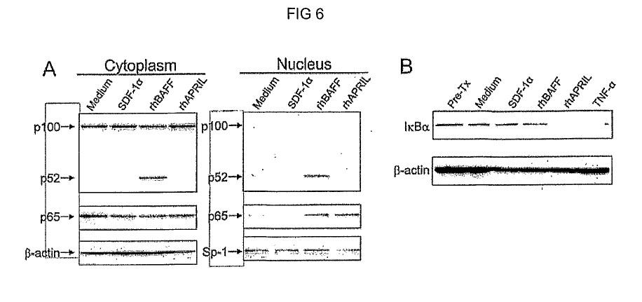

Because NLC express BAFF, APRIL, and SDF-1α, we examined whether these factors together could support CLL B cell survival better than either factor alone. The viability of isolated CLL B cells was highest when co-cultured with NLC (FIG. 6). However, isolated CLL B cells co-cultured with rhBAFF plus SDF-1α, or rhAPRIL plus SDF-1α, had a significantly greater viability than that of CLL B cells cultured with any one factor alone (FIG. 5). Collectively, these data support the notion that BAFF or APRIL promotes leukemia cell survival via a mechanism(s) independent of that used by SDF-1α.

FIG. 5 shows the effect of rhBAFF, rhAPRIL, and/or SDF-1α on CLL-cell Survival

CLL B cells were cultured with (open squares) or without (closed squares) NLC. SDF-1α (closed circles) rhAPRIL (closed diamonds) at 500 ng/ml, rhBAFF (closed triangles) at 50 ng/ml or both (open diamonds) were added to wells without NLC at day 0. Also SDF-1α and rhBAFF (open diamonds) or SDF-1α and rhAPRIL (open circles) were added to the cultures without NLC. The mean viability ±S.E. of replicate wells was determined for each time point indicated on the horizontal axis. A representative example of three different CLL patients is presented.

Effects of rhBAFF, rhAPRIL, or SDF-1α on Signaling Pathways in CLL B Cells

We examined the intracellular signaling pathways stimulated by rhBAFF, rhAPRIL, or SDF-1α at concentrations that can promote CLL B cell survival in vitro. Prior studies indicated that BAFF could induce activation of the NF-κB2 in normal B cells, a pathway that appears critical for the growth and/or survival of normal B cells. Such activation involves processing of p100 to p52 with subsequent translocation of p52 to the nucleus. We found that rhBAFF could induce activation of NF-κB2 also in CLL B cells (FIG. 6A). In contrast, we did not observe activation of NF-κB2 in CLL cells treated with rhAPRIL or SDF-1α, even at concentrations that could support CLL cell survival in vitro. Both rhBAFF and rhAPRIL, however, induced degradation of the inhibitor of kappa B (IκBα) and translocation of p65 to the nuclear fraction, indicating activation of the classical NF-κB pathway (FIG. 6B). SDF-1α, on the other hand, did not have this activity (FIG. 6).

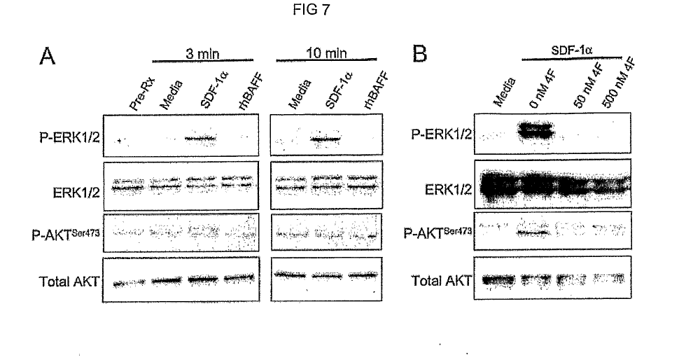

We also examined for phosphorylation and activation of AKT, which prior studies found also could enhance CLL B cell survival. In contrast to SDF-1α, we found that rhBAFF or rhAPRIL could not induce phosphorylation of p44/42 mitogen-activated phosphokinase (MAPK, ERK1/2) or activation of AKT in CLL B cells, even at concentrations that could promote CLL B cell survival in vitro (FIG. 7, and data not shown).

However, SDF-1α not only induced phosphorylation of ERK1/2, as noted previously, but also induced phosphorylation of AKT at Ser473 in isolated CLL B cells (FIG. 7A). The capacity of SDF-1α to induce CLL-cell phosphorylation of ERK1/2 and AKT at Ser473 could be blocked by 4F-benzoyl-TE14011 (4F), a specific CXCR4 antagonist (FIG. 7B).

FIG. 7(A) shows CLL B cells cultured for 3 or 10 minutes with SDF-1α (200 ng/ml), rhBAFF (50 ng/ml), or media, as indicated above the sample lanes. Cell lysates were prepared and analyzed by immunoblot using antibodies specific for phosphorylated ERK1/2 (P-ERK1/2), ERK1/2, phosphorylated AKT (P-AKTSer473), or AKT, as indicated on the left-hand margin. Equal loading in the lanes was evaluated by stripping the blot and probing again with anti-ERK1/2 and an anti-AKT antibody. Five different CLL B cells gave similar results. In FIG. 7(B) the CLL cells were stimulated for 3 minutes with either media (far left lane) or SDF-1α (200 ng/ml) (right three lanes). For samples treated with SDF-1α we included the CXCR4 antagonist 4F-benzoyl-TE14011 (4F) at 0 nM, 50 nM, or 500 nM, respectively. The samples were analyzed and the results presented as noted in FIG. 7A.

NLC, BAFF, or APRIL, but not SDF-1α, can Induce CLL-Cell Expression of Mcl-1

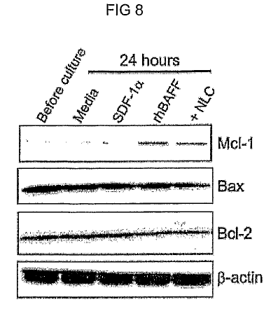

To examine mechanisms that might account for the effects on leukemia-cell survival, we evaluated for the expression of pro-apoptotic and anti-apoptotic proteins in CLL B cells following culture with or without NLC or with either rhBAFF or SDF-1α. We did not observe significant changes in the levels of Bcl-2, Bax, or Bcl-xL expressed by isolated CLL B cells in any of the short-term culture conditions used (FIG. 8, and data not shown). On the other hand, CLL B cells co-cultured with NLC, rhBAFF, or rhAPRIL were induced to express increased levels of Mcl-1 (FIG. 8 and data not shown). In contrast, SDF-1α could not induce isolated CLL B cells to express higher levels of Mcl-1, even at concentrations that could protect CLL-cell survival in vitro (FIG. 8, and data not shown).

Increasing attention is being focused on cells and factors of different microenvironments that contribute to CLL cell survival. Such accessory cells include marrow stromal cells, follicular dendritic cells, and NLC. Defining the mechanisms whereby these cells contribute to the survival of CLL cells potentially could identify novel targets for treatment of this disease.

In this study, we found that NLC express high levels of BAFF and APRIL, two factors of the TNF family that play an important role in maintaining the survival of mature B cells. Because NLC are derived from CD14+ cells, expression of BAFF by NLC was anticipated, as this factor originally was found expressed by myeloid lineage cells, such as monocytes, macrophages, or dendritic cells. Moreover, we found that CD14+ cells accounted for most of the BAFF mRNA found in the blood mononuclear cells of patients with CLL and, on a cell-per cell basis, contained approximately 30-fold more BAFF mRNA than did CLL B cells, which prior studies found could also express this B-cell survival factor. From the studies reported here, it is appears that such CD14+ cells maintain high level expression of BAFF, even after they differentiate into NLC upon co-culture with CLL B cells in vitro.

In contrast, NLC expressed significantly more APRIL than newly isolated CD14+ blood cells, which in turn contributed little to the APRIL mRNA detected in the blood mononuclear cells of patients with CLL. Moreover, the low-to-negligible amount of APRIL mRNA detected in CD14+ blood mononuclear cells appeared less than that expressed by CLL B cells, or even normal B cells. In contrast, CD14+ myeloid cells in the secondary lymphoid tissues of patients with non-Hodgkin's lymphomas, including CLL, apparently express high-levels of BAFF and APRIL. Conceivably, such cells may include CD14+ cells that already have differentiated into NLC in vivo.

We investigated whether BAFF and/or APRIL on NLC could contribute to their capacity to promote leukemia cell survival in vitro. Previous studies showed BCMA-Fc could impair leukemia-cell viability over time when this decoy receptor was added to isolated leukemia cells. However, we did not observe this effect on the viability of CLL B cells cultured without NLC, even at concentrations of BCMA-Fc of 30 μg/ml (data not shown). The reason for the discrepancy between our data and others is not clear. Instead, BCMA-Fc significantly impaired the viability of CLL B cells cultured with NLC (FIG. 4A). However, BAFF-R:Fc, which only can inhibit BAFF interactions with BAFF-R, failed to impair the viability of CLL cells that were cultured either with or without NLC, implying that APRIL may play an important role in the protective effect(s) of NLC on CLL cell survival. Although the studies in knock-out mice showed that APRIL appeared to be dispensable for developing normal immune systems, a recent study by Planelles found that APRIL may play a role in the pathogenesis of B1-cell malignancies, namely CLL. In this light, strategies that only interfere with BAFF/BAFF-R interactions may not be sufficient to affect CLL cell viability in vivo

Previously, we reported that NLC also express SDF-1α, a chemokine that can trigger phosphorylation of p44/42 MAPK ERK1/2 and enhance CLL cell survival in vitro. Although some studies have suggested that the ERK pathway might not be involved in preventing spontaneous apoptosis of CLL B cells, suppression of ERK activity is seen in CLL B cells undergoing drug induced apoptosis, suggesting that this pathway is important for survival of CLL B cells.

Since SDF-1α had an additive effect on the viability of isolated CLL cells cultured with BAFF and/or APRIL (FIG. 5), we reasoned that BAFF or APRIL might promote CLL cell survival via a pathway(s) that is distinct from that of SDF-1α. Consistent with this notion, we found that SDF-1α, in addition to its noted capacity to induce phosphorylation of ERK1/2 MAPK, could induce CLL B cells activation of phosphatidylinositol 3-kinase (PI3K) AKT (FIG. 7), a pathway that is essential for the survival of CLL B cells. These findings are consistent with those of others who found that SDF-1α could induce activation of AKT in other types of cells besides leukemia B cells. Recently, Moreaux and colleagues reported that addition of exogenous BAFF to myeloma cells induced late activation of both ERK1/2 and AKT, but the direct influence of BAFF on these two pathways was not resolved. In the study presented here, it appears that neither pathway is activated in CLL cells by rhBAFF or rhAPRIL, indicating that these factors must use other mechanisms to protect CLL B cells from spontaneous apoptosis.

Some TNF superfamily proteins like BAFF trigger their functions by activating NF-κB. Two main pathways—the canonical and alternative pathway—regulate the activity of NF-κB. Activation of the canonical pathway results from degradation of the inhibitor of NF-κBα (IκBα), which is induced upon its phosphorylation by the beta subunit of the IκB kinase (IKK) complex, IKKβ. This leads to nuclear translocation of active NF-κB heterodimers (that are composed of p65, c-Rel or p50) where they can effect changes in gene expression. As noted for lymphoma or CLL B cells, concentrations of rhBAFF or rhAPRIL required for optimal enhancement of CLL cell survival also induced degradation of IκBα and translocation of p65 to the nucleus, indicating that either factor can activate the canonical NF-κB pathway. Activation of the canonical NF-κB pathway in normal B cells appears secondary to the capacity of BAFF or APRIL to interact with BCMA, or BCMA and/or TACI, respectively.

Alternative pathway activation results from processing of NF-κB2 p100 to p52, which is triggered by the phosphorylation of NF-κB2 p100 by the alpha subunit of the IKK complex, namely IKKα. This allows for nuclear translocation of p52 along with RelB, where this complex can influence expression of genes that are distinct from those regulated by the canonical NF-κB pathway. We noted that rhBAFF, but not rhAPRIL or SDF-1α, could induce degradation of p100 to p52 and translocation of p52 to the nucleus. Because the BAFF-R interacts with BAFF, but not APRIL, the selective activation of p100 processing by BAFF suggests that the BAFF-R may be distinct from BCMA or TACI in its capacity to activate the alternative NF-κB pathway in CLL B cells. This is similar to the interaction of BAFF with its receptor on normal B cells, which also promotes processing of NF-κB2. Moreover, studies have shown that IKKα is required for B cell maturation and formation of secondary lymphoid organs. However, because treatment of co-cultures of CLL cells and NLC with BAFF-R:Fc failed to inhibit the protective effect of NLC on leukemia cell survival, it appears that activation of the canonical pathway may obviate the requirement for activation of the alternative NF-κB pathway in CLL to promote leukemia cell survival, at least in the in vitro culture conditions used in this study.

Finally, we evaluated for expression of Bcl-2-family-member proteins that can influence the resistance or sensitivity of CLL cells to apoptosis. Prior studies found that BAFF can up-regulate expression of Bcl-2 in most B cells. BAFF induced up-regulation of Bcl-2 was less apparent in CLL B cells, possibly secondary to the constitutive high-level expression of this anti-apoptotic protein in this leukemia. However, we found that rhBAFF, rhAPRIL, or NLC could induce CLL B cells to express high-levels of Mcl-1 (FIG. 8, and data not shown). Like Bcl-2, Mcl-1 also appears to play a role in the resistance of CLL B cells to drug induced apoptosis, and patients with CLL who fail to achieve complete remission after chemotherapy tend to have high levels of Mcl-1. There are several reports that AKT or ERK1/2 regulate the expression of Mcl-1 in various types of cells. On the other hand, O'Connor reported that the persistence of plasma cells in mice was associated with a BAFF-mediated up-regulation of Mcl-1. In the present study, we found that rhBAFF or rhAPRIL, which did not activate AKT or ERK1/2, up-regulated Mcl-1 in CLL B cells. However, saturating amounts of BCMA-Fc or BAFF-R:Fc that could inhibit rhBAFF-induced expression of Mcl-1 failed to block the capacity of NLC to enhance expression of Mcl-1 in CLL B cells (data not shown), suggesting that NLC-associated factors other than BAFF and APRIL also may induce expression of this anti-apoptotic protein in CLL cells. In any case, we found that SDF-1α, which can activate AKT or ERK1/2 in CLL cells, was unable to induce CLL cells to express Mcl-1 (FIG. 8). As such, these data suggest that BAFF up-regulates expression of Mcl-1 in CLL B-cell via a pathway(s) distinct from that involving activation of MAPK or AKT.

Whereas isolated CLL B cells undergo apoptosis when cultured alone, the addition of rhBAFF, rhAPRIL, and/or SDF-1α to the CLL B cells significantly enhanced their viability (FIG. 5), as noted previously. Nevertheless, the viability of CLL cells cultured with SDF-1α and rhBAFF and/or rhAPRIL still was not as high as that seen when CLL B cells were cultured with NLC, suggesting that yet additional NLC factors are involved in promoting leukemia-cell survival. In this regard, it is noteworthy that Deaglio and colleagues recently found that NLC also express high-levels of CD31 and plexin-B1, which also can contribute in part to the capacity of NLC to sustain CLL cell viability. Conceivably, strategies that can target one or more of the mechanisms whereby NLC sustain CLL cell survival could have therapeutic potential for patients with this disease.

Canonical Pathway NF-κB1 and the Alternative Pathway NF-κB2

The B cell-activating factor of tumor necrosis factor (TNF) family (BAFF), also known as BlyS, TALL-1, zTNF4, or THANK) is a potent regulator of normal B cell development and function. A proliferation-inducing ligand (APRIL, also termed TALL-2 or TRAD-1), which is also a member of TNF family, shares significant homology with BAFF. APRIL has been found to stimulate tumor cell growth as well as proliferation of primary lymphocytes. Both BAFF and APRIL bind two receptors of the TNF superfamily, B-cell maturation antigen (BCMA) and transmembrane activator or the calcium modulator and cyclophilin ligand-interactor (TACI). BAFF, but not APRIL, binds a third receptor named BAFF receptor (BAFF-R or BR3). BCMA, TACI, and BR3 are expressed on normal B lymphocytes.

The neoplastic B cells in chronic lymphocytic leukemia (CLL) also express these receptors BAFF and APRIL, which, when ligated, can promote CLL cell survival in vitro. Furthermore, “nurselike cells” (NLC), which can protect CLL cells in vitro and presumably in vivo, express high-levels of BAFF and APRIL, accounting in part for their capacity to promote CLL cell survival in a paracrine fashion. Kern and colleagues also found that CLL cells themselves may express BAFF and/or APRIL, suggesting that these factors also can function in an autocrine fashion to promote leukemia-cell survival. As such, understanding of the mechanisms whereby BAFF and/or APRIL support the CLL survival could lead to development inhibitors to BAFF and/or APRIL signaling that could lead to new and more effective treatments for patients with this disease.

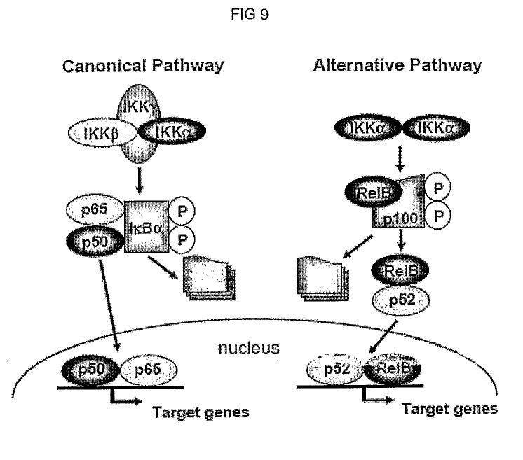

Many members of the TNF super-family trigger activation of nuclear factor of kappa B (NF-κB). Recent studies have revealed that two NF-κB pathways, the canonical pathway (NF-κB1) and the alternative pathway (NF-κB2), regulate the activity of NF-κB (FIG. 9). Activation of the canonical pathway proceeds through degradation of the inhibitor of NF-κBα (IκBα), which is induced upon its phosphorylation by the beta subunit of the IκB kinase (IKK) complex (IKKβ). Degradation of IκBα leads to nuclear translocation of active NF-κB heterodimers (comprised of p50, p65, and/or c-Rel) where they can affect changes in gene expression. Activation of the alternative NF-κB2 pathway results from processing of NF-κB2/p100 to p52, which is triggered by the phosphorylation of NF-κB2/p100 by the alpha subunit of the IKK complex (IKKα). This allows for nuclear translocation of p52 along with RelB, where they together can influence expression of genes that are distinct from those regulated by the canonical NF-κB1 pathway. Studies have shown that NF-κB1 is constitutively activated in CLL cells and sustained activation of NF-κB is critical for the survival of CLL cells. However, the relative contribution of each NF-κB pathway in promoting CLL cell survival has not been described. We examined which NF-κB pathways are stimulated in CLL cells by BAFF or APRIL and investigated the relative contribution of each pathway to BAFF and/or APRIL-induced leukemia-cell survival.

FIG. 9 provides a schematic of signaling pathway of NF-κB. There are two distinct NF-κB activating pathways, the canonical and alternative pathway. Activation of the canonical pathway depends on the three-subunit IKK hotocomplex, which phosphorylates IκBαto induce its degradation. This leads to nuclear translocation of active NF-κB heterodimers (that are composed of p65, c-Rel or p50) where they can effect changes in gene expression. Activation of the alternative pathway depends on IKKα homodimers, which induce processing of p100 to p52. This allows for nuclear translocation of p52 along with RelB, where this complex can influence expression of genes that are distinct from those regulated by the canonical NF-κB pathway.

Expression of BCMA, TACI, and BR3 on CLL B cells

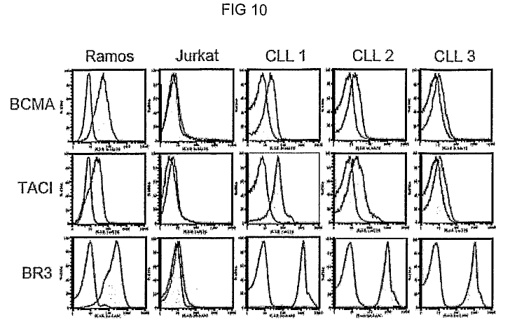

We examined for surface expression of BCMA, TACI, and BR3 on CLL B cells using flow cytometry. Of eleven samples tested we found 8 expressed detectable BCMA, 9 expressed detectable TACI, and 11 expressed BR3, consistent with earlier findings. Three representative samples are shown in FIG. 10. Thus, CLL B cells typically express all three receptors for BAFF or APRIL. Because exogenous BAFF and APRIL can improve the viability of CLL cells in vitro, signaling through these receptors can enhance CLL cell survival.

FIG. 10 depicts the expression of BCMA, TACI, and BR3 on CLL B cells. B cells from CLL patients were tested using FACS for surface expression of BCMA, TACI, and BR3 by labeling with specific primary and secondary antibodies (gray histogram) or isotype controls (open histograms). Representative histograms of 3 CLL patients were shown. CLL B cells express at their surfaces the three receptors for BAFF or APRIL.

Effects of rhBAFF or rhAPRIL on NF-κB Signaling Pathways in CLL B Cells

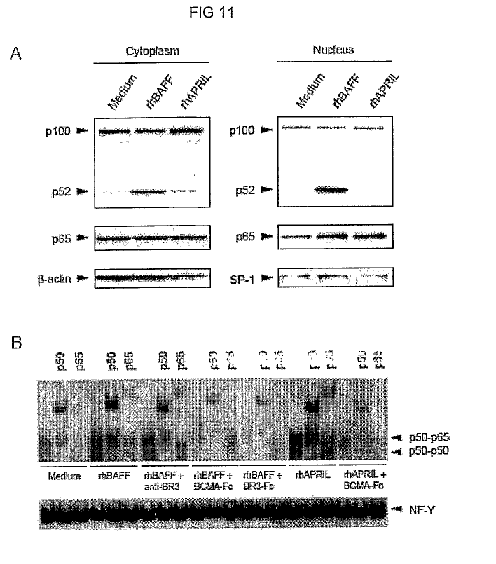

We examined for activation of NF-κB signaling pathways in CLL cells treated with recombinant human BAFF (rhBAFF) or rhAPRIL at concentrations that could promote CLL B cell survival in vitro. Prior studies indicated that BAFF could induce activation of the NF-κB2/p100 in normal B cells. Such activation involves processing of p100 to p52 with subsequent translocation of p52 to the nucleus (FIG. 9). We found that rhBAFF could induce translocation of p52 to the nucleus also in CLL B cells (FIG. 11A), demonstrating activation of the NF-κB2 pathway. In contrast, we did not observe translocation of p52 to the nucleus in CLL cells treated with rhAPRIL, even at concentrations that could support CLL cell survival in vitro. Both rhBAFF and rhAPRIL, however, induced translocation of p65 to the nucleus, indicating that each could activate the canonical NF-κB1 pathway in CLL cells (FIG. 11A). Activation of the canonical NF-κB1 by rhBAFF or rhAPRIL was verified using the Electrophoretic Mobility Shift Assay (EMSAs). Nuclear extracts prepared from CLL cells cultured with rhBAFF or rhAPRIL contained increased amounts of proteins capable of binding NF-κB consensus motifs that experienced a supershift when pre-incubated with anti-p50 or anti-p65 antibodies (FIG. 11B). Nuclear extracts of CLL cells treated with rhBAFF or rhAPRIL in the presence of soluble BCMA (BCMA-Fc), which can bind BAFF and/or APRIL and preclude them from binding their receptors on the CLL cell surface, had less NF-κB1 binding activity. Nuclear extracts of CLL cells treated with rhBAFF in the presence of soluble BR3 (BR3-Fc) also contained lower amounts of NF-κB1 binding activity. However, nuclear extracts of CLL cells treated with rhBAFF and anti-BR3 antibody, which can bind to BR3 and block BAFF binding to BR3 but not to BCMA or TACI, contained amounts of NF-κB1 binding factors similar to that of extracts prepared from CLL cells treated with rhBAFF alone (FIG. 11B). These results suggest that for CLL B cells signaling through BR3, but not from BCMA or TACI, could activate the alternative NF-κB2/p100 pathway, whereas signaling through BCMA and/or TACI could activate the canonical NF-κB1 pathway.

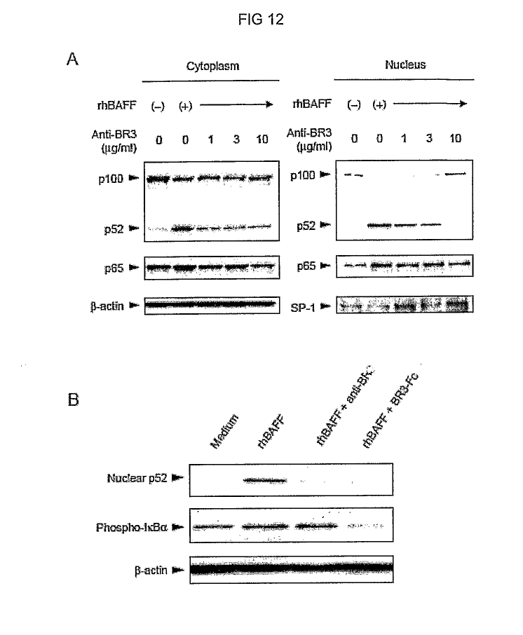

FIG. 12 depicts the activation of NF-κB in CLL B cells by rhBAFF or rhAPRIL. CLL B cells were cultured with or without rhBAFF (50 ng/ml), rhAPRIL (500 ng/ml), BCMA-Fc (10 μg/ml), BR3-Fc (10 μg/ml) or anti-BR3 (10 μg/ml) for 24 hours. Cytoplasmic and nuclear extracts were prepared as described in “material and methods”. (A) Immunoblot analysis with anti-p100 or anti-p65 antibodies. We evaluated for equal loading in each lane by stripping the blot and probing it again with antibodies specific for β-actin (for cytoplasmic extracts) or SP-1 (for nuclear extracts). Translocation of p65 to the nucleus was seen in CLL cells treated with rhBAFF or rhAPRIL. In contrast, translocation of p52 was observed only in CLL cells treated with rhBAFF. (B) EMSAs of CLL cells and supershift with ant-p50 or anti-p65 antibodies. We evaluated for equal loading in each lane by NF-Y. Up-regulation of NF-□B binding to DNA was seen in CLL cells cultured with rhBAFF and rhAPRIL. In the presence of BCMA-Fc or BR3-Fc, CLL cells down-regulated NF-□B binding to DNA. However, anti-BR3 antibody could not inhibit NF-□B binding to DNA up-regulated by rhBAFF.

To verify the selective capacity of BR3 to activate the alternative NF-κB pathway, CLL cells were cultured with rhBAFF and increasing concentrations of anti-BR3 antibody. CLL cells cultured with rhBAFF without anti-BR3 were stimulated to effect nuclear translocation of both p52 and p65. Addition of anti-BR3 inhibited BAFF from inducing activation of the alternative pathway. Anti-BR3 at 10 μg/ml could completely inhibit BAFF induced translocation of p52, but not p65 (FIG. 12A). BR3-Fc inhibited both p52 translocation to the nucleus and phosphorylation of IκBαinduced by rhBAFF. Anti-BR3, however, could not inhibit phosphorylation of IκBα(FIG. 12B). These data indicate that signaling via BR3 is necessary and sufficient to activate the alternative NF-κB2/p100 pathway in CLL cells.

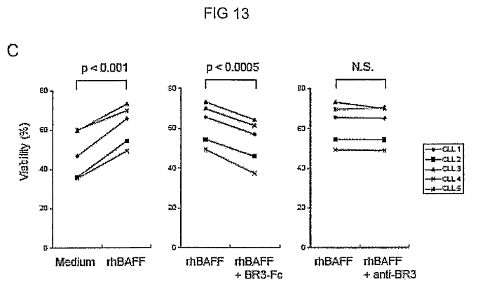

CLL cells were then cultured with rhBAFF and anti-BR3 or BR3-Fc to examine the role of the alternative pathway of NF-κB in the survival of CLL cells. Addition of BR3-Fc to CLL cells cultured with rhBAFF inhibited the anti-apoptotic effect of rhBAFF. On the other hand, anti-BR3 at the concentration that could completely block activation of the alternative NF-κB2/p100 pathway, did not impair the capacity of rhBAFF to enhance CLL cells survival in vitro (FIG. 13C). These results suggest that signaling through the alternative NF-κB2/p100 pathway does not contribute significantly to CLL cell survival.

FIG. 13 depicts the blocking the alternative NF-□B pathway with anti-BR3 antibody. (A) CLL B cells were cultured with or without rhBAFF (50 ng/ml) and the indicated concentration of anti-BR3 for 24 hours. Cytoplasmic and nuclear extracts were prepared as described in “material and methods” for immunoblot analysis. The protein content was normalized to 25 □g for cytoplasmic fraction and 12.5 μg for nuclear fraction. Translocation of p52 and p65 to the nucleus were seen in CLL cells treated with rhBAFF. Anti-BR3 at 10 □g/ml could completely inhibit p52 translocation to the nucleus induced by rhBAFF. (B) CLL B cells were cultured with or without rhBAFF (50 ng/ml) and anti-BR3 (10 μg/ml) or BR3-Fc (10 μg/ml) for 24 hours. Total cell lysates were prepared as described in “material and methods”. BR3-Fc inhibited both p52 translocation to the nucleus and phosphorylation of I□B□ induced by rhBAFF. Anti-BR3 could inhibit p52 translocation but not phosphorylation of IκBα (C) CLL B cells were cultured with or without rhBAFF (50 ng/ml) and anti-BR3 (10 μg/ml) or BR3-Fc (10 μg/ml) for 48 hours. Results are viability of samples from each of 5 patients. The viability of CLL cells cultured with both rhBAFF and BR3-Fc was significantly lower than that of CLL cells cultured with rhBAFF alone (P<0.0005; Student paired t test). Anti-BR3 did not impair survival of CLL cells cultured with rhBAFF.

Blocking the Canonical NF-κB Pathway with IKKβ Inhibitor

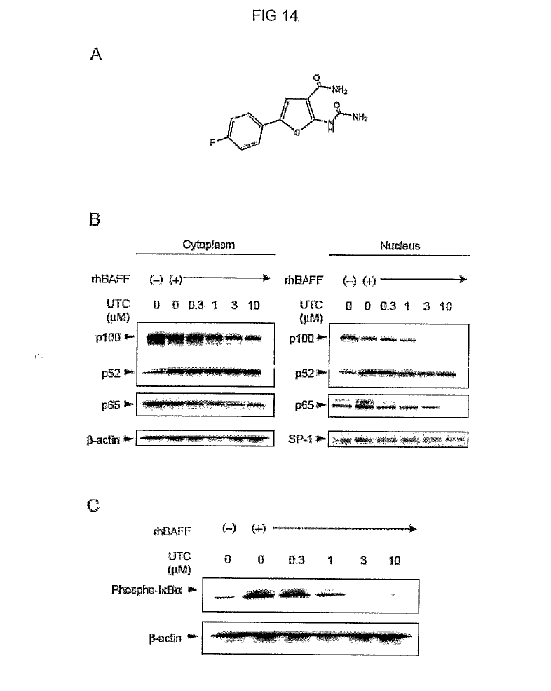

Activation of the canonical NF-κB pathway depends upon IKKβ-dependent phosphorylation-induced degradation of IκBα. Several compounds or natural products have been found to inhibit IKKβ, the subunit responsible for phosphorylation of IκBα. We synthesized one such IKKβ inhibitor, 5-(4-fluorophenyl)-2-ureido-thiophene-3 carboxylic acid amide (UTC), to block the canonical NF-κB pathway in CLL cells (FIG. 14A). One of skill in the art will recognize that other IKKβ inhibitors may be tested and used to treat CLL according to the teachings of the present invention. Such inhibitors include those disclosed in: Karin, M., Y. Yamamoto, and Q. M. Wang. 2004. The IKK NF-kappa B system: a treasure trove for drug development. Nat Rev Drug Discov 3:17-26. Hideshima, T., D. Chauhan, P. Richardson, C. Mitsiades, N. Mitsiades, T. Hayashi, N. Munshi, L. Dang, A. Castro, V. Palombella, J. Adams, and K. C. Anderson. 2002. NF-kappa B as a therapeutic target in multiple myeloma. J Biol Chem 277:16639-16647. Lam, L. T., R. E. Davis, J. Pierce, M. Hepperle, Y. Xu, M. Hottelet, Y. Nong, D. Wen, J. Adams, L. Dang, and L. M. Staudt. 2005. Small molecule inhibitors of IkappaB kinase are selectively toxic for subgroups of diffuse large B-cell lymphoma defined by gene expression profiling. Clin Cancer Res 11:28-40. Frelin, C., V. Imbert, E. Griessinger, A. C. Peyron, N. Rochet, P. Philip, C. Dageville, A. Sirvent, M. Hummelsberger, E. Berard, M. Dreano, N. Sirvent, and J. F. Peyron. 2005. Targeting NF-kappaB activation via pharmacologic inhibition of IKK2-induced apoptosis of human acute myeloid leukemia cells. Blood 105:804-811. Each reference is incorporated herein by reference in its entirety for all purposes.

First we examined whether UTC could block activation of the canonical NF-κB1 pathway in CLL cells. CLL cells were pre-incubated with or without varying concentrations of UTC for 1 hour. The treated cells then were cultured with or without rhBAFF for 24 hours. UTC inhibited BAFF induced nuclear translocation of p65, but not p52 (FIG. 14B). UTC also inhibited phosphorylation of IκBα (FIG. 14C). These data indicate that UTC can block BAFF-induced activation of the canonical NF-κB1 pathway, but not the alternative NF-κB2/p100 pathway.

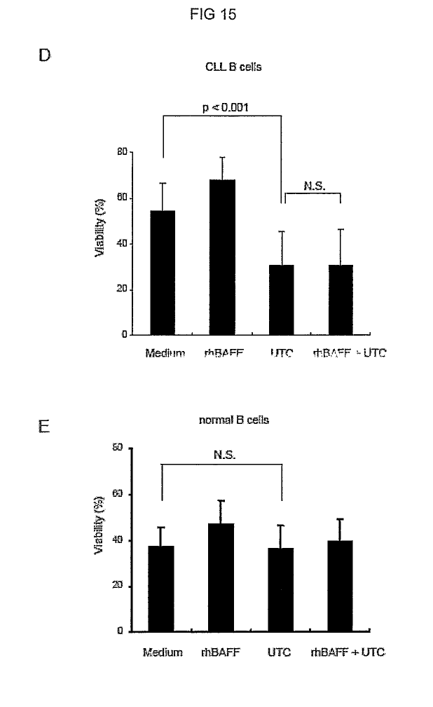

CLL cells were cultured with or without rhBAFF and UTC to determine whether blocking the canonical NF-κB1 pathway could impair the capacity of rhBAFF to enhance the survival of CLL cells in vitro. Treatment of CLL cells with UTC significantly inhibited the capacity of rhBAFF to support CLL cell survival (FIG. 15D). However, UTC did not have any effect on survival of isolated normal B cells of healthy donors, although could partially block the pro-survival effect of exogenous rhBAFF on normal B cells in vitro (FIG. 15E). These findings suggest that activation of the canonical NF-κB1 pathway may play a more important role in promoting the survival of CLL cells than that of normal B cells.

FIG. 15 depicts the blocking of the canonical NF-□B pathway with IKK□ inhibitor. (A) The chemical structure of the Ikk□ inhibitor, 5-(4-fluorophenyl)-2-ureido-thiophene-3 carboxylic acid amide (UTC) (B) CLL cells were pre-incubated with or without various concentrations of UTC for 1 hour. Then cells were cultured with or without rhBAFF (50 ng/ml) for 24 hours and cytoplasmic and nucleus cell lysates were recovered. The protein content was normalized to 25 □g for cytoplasmic fraction and 12.5 μg for nuclear fraction. UTC inhibited BAFF induced nucleus translocation of p65, but not p52. (C) Total cell lysates of CLL cells were prepared after the same treatment as above. UTC inhibited BAFF induced phosphorylation of IκBα. (D) CLL cells were cultured with or without rhBAFF (50 ng/ml) and UTC (10 μM) for 48 hours. Results are viability of samples from each of 8 patients. The viability of CLL cells cultured with UTC was significantly lower than that of CLL cells cultured with medium alone (P<0.001; Bonferroni t test). Anti-apoptotic effect of BAFF wasn't seen when CLL cell were cultured with UTC. (E) Isolated normal B cells of healthy donors were cultured with or without rhBAFF (50 ng/ml) and UTC (10 μM) for 48 hours. Results are viability of samples from each of 8 donors. There was no significant difference between the viability of normal B cell cultured with and without UTC, although it partially blocked the effect of exogenous rhBAFF.

Blocking the Canonical NF-κB Pathway with Transfection of SR-IκBα

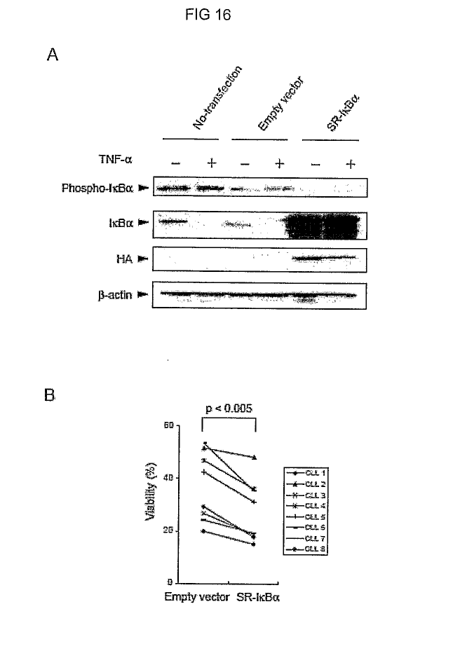

Conceivably UTC also could affect signaling pathways other those leading to activation of the canonical NF-κB1 pathway. If so, then the capacity of UTC to inhibit the survival promoting effects of rhBAFF on CLL cells may not be due to its capacity to block activation of the canonical pathway. To rule out this possibility, we transfected super-repressor IκBα (SR-IκBα) into CLL cells using a plasmid expression vector. SR-IκBα encodes a mutant IκBαin which the serines at positions 32 and 36 are replaced by alanines. As such, this mutant form of IκBα can bind to p50 and p65, but cannot be phosphorylated upon cellular activation and therefore resists proteolytic degradation. In control studies we transfected HeLa cells with either a control plasmid expression vector (pcDNA3) or SR-IκBα and then monitored the cells for degradation of IκBαfollowing treatment with recombinant TNFα. Phosphorylation and degradation of IκBα were observed in TNFα treated HeLa cells that either were not transfected or transfected with the control pcDNA3 vector. On the other hand, examination of HeLa cells transfected with SR-IκBA revealed persistent, high-level expression o IκBαthat was unaffected by treatment with TNF-α (FIG. 16A).

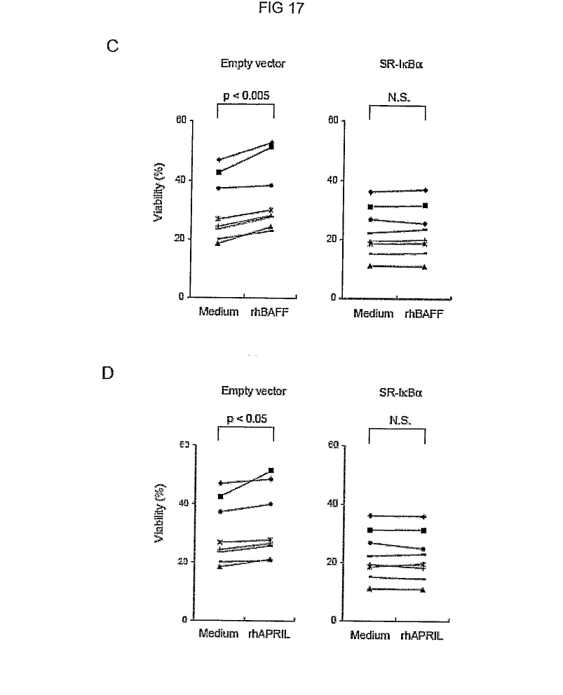

We transfected CLL cells from each of 8 patients with SR-IκBα, the control pcDNA3 vector, or a pcDNA3 vector encoding the green fluorescent protein (GFP). Transfection efficiencies ranged from 30-55%, as assessed by flow cytometry of cells transfected with the GFP expression plasmid. In all samples tested, the CLL cells transfected with SR-IκBα had lower viabilites following transfection than that of control treated cells or CLL cells transfected with any of the other two control vectors at 24 hours after transfection (FIG. 16B). Moreover, treatment of CLL cells with rhBAFF or rhAPRIL following transfection significantly enhanced the viability of the cells transfected with the control expression plasmid, but had no effect on CLL cells transfected with SR-IκBα (FIG. 17C, D). These results support the notion that activation of the canonical NF-κB1 pathway plays a critical role in promoting CLL cell survival following treatment with BAFF or APRIL.

FIG. 17 depicts the blocking of the canonical NF-κB pathway with transfection of SR-IκBα. (A) HA-tagged SR-IκBα(S32A/S36A) or empty pcDNA3 vector were transfected into HeLa cells using Lipofectin Plus (Invitrogen). Twenty-four hours after transfection, cells were cultured in serum free medium for 3 hours, and then stimulated with recombinant TNF-α (50 ng/ml) for 30 minutes and total cell lysates were obtained for immunoblot analysis. Phosphorylation and degradation of IκB were seen in non-transfected HeLa cells and empty vector transfected HeLa cells when these cells were stimulated with TNF-α. On the other hand, phosphorylation of IκB was not seen in SR-IκBα transfected HeLa cells. High expression of IκBα was seen and it was not degraded with TNF-α stimulation. (B) Each sample from 8 CLL patients was divided into two and transfected with either empty vector or SR-IκBα, using Amaxa nucleofection technology (Amaxa). The viability after 24 hours of transfection of each 8 patients is shown. In all patients, SR-I□B□ transfected CLL cells underwent apoptosis more readily than control cells transfected with empty vector at 24 hours after transfection (P<0.005; Student paired t test). (C, D) CLL cells were transfected with empty vector or SR-IκBα. Four hours after transfection, these cells were cultured with or without rhBAFF (50 ng/ml) or rhAPRIL (500 ng/ml) for 24 hours. Results are viability of samples from each of 8 patients. In empty vector transfected cells, the viability of CLL cells cultured with rhBAFF or rhAPRIL was significantly higher than that cultured with medium alone (P<0.005, P<0.05, respectively; Student paired t test). The survival of SR-I□B transfected cells could not be enhanced by rhBAFF or rhAPRIL.

BAFF has been reported to be a potent regulator of normal B cell development and function. BAFF also plays an important role in the resistance to apoptosis of malignant B cells, such as CLL, lymphoma, and myeloma cells. APRIL has been found to stimulate tumor cell growth as well as proliferation of primary lymphocytes. Moreover, a recent study found that transgenic mice overexpressing APRIL develop a clonal expansion of B1 lymphocytes similar to that seen in CLL. Recently, we reported that “nurselike cells” express both BAFF and APRIL and could promote CLL-cell survival in a paracrine manner. As such, strategies that can block leukemia-cell signaling induced BAFF and APRIL may disrupt the support of the leukemic cells provided by their microenvironment. Therefore, we examined the mechanism whereby BAFF and/or APRIL could support leukemia cell survival in vitro.

Both BAFF and APRIL are known to trigger their functions by activating NF-κB. However, NF-κB signaling pathways from their receptors, namely BCMA, TACI, and BR3, have not been well documented in CLL cells. In this study, we showed that rhBAFF, but not rhAPRIL could induce degradation of p100 to p52 and translocation of p52 to the nucleus, indicating activation of the alternative NF-κB2/p100 pathway (FIG. 11A). Because BR3 interacts with BAFF, but not APRIL, the selective activation of the alternative NF-κB2/p100 pathway by BAFF indicates that signaling via BR3 is distinct from that through BCMA or TACI. This is similar to the interaction of BAFF with BR3 on normal B cells, which also promotes processing of NF-κB2/p100. Morrison and colleagues reported that this specific function of BR3 is mediated by a sequence motif, PVPAT, which is homologous to the TRAF-binding site (PVQET) present in CD40. They also showed that BR3 preferentially induced the alternative NF-κB2/p100 pathway. In our studies, we found that anti-BR3 could not block BAFF-induced activation of the canonical NF-κB1 pathway (FIG. 12A, B). This is in contrast to a recent report suggesting that signaling through BR3 could activate both the canonical NF-κB1 and the alternative NF-κB2/p100 pathways. One explanation for this might be that the canonical NF-κB1 pathway might be sufficiently activated through the other BAFF receptors, BCMA and TACI in CLL cells. This explanation is supported by the observation that BR3-Fc, which can bind to BAFF and block BAFF binding to its receptors, could inhibit both the canonical NF-κB1 and the alternative NF-κB2/p100 pathways (FIG. 12B).

Investigators have shown that some tumor cells that did not bind BAFF responded to APRIL. These findings suggest that APRIL may have a specific receptor (APRIL-R) expressed on these tumor cells that cannot bind BAFF. It is not clear whether such a hypothetical APRIL-R also is expressed on CLL cells. If so, then the studies presented here suggest that such a specific APRIL-R does not activate the alternative NF-κB2/p100 pathway in these leukemia cells (FIG. 11A).

Studies have shown that IKKα, which is involved in both the canonical and the alternative NF-κB pathway, is essential for B cell maturation and formation of secondary lymphoid tissues in mice. IKKβ, which is involved in the canonical NF-κB pathway, also is reported to be required for the survival and proliferation of normal blood B cells in mice. It was reported that BR3-knockout mice displayed strongly reduced numbers of late transitional and follicular B cells and were essentially devoid of marginal zone B cells. Over-expression of the anti-apoptotic protein Bcl-2 rescued mature B cell development in these mice. In addition, NF-κB2/p100 deficient mice also were reported to have a marked reduction in B cell numbers. These findings indicate that BR3 mediates a survival signal in B cells, and NF-κB2/p100, which is involved in the alternative NF-κB pathway, has an important role in the maintenance of the population of normal B cell population in mice. This may be in contrast to what governs survival of neoplastic CLL B cells.

Instead, CLL cells have have high constitutive levels of NF-κB1 activity compared with non-malignant, normal human B cells. Moreover, sustained activation of NF-κB1 is critical for the survival of CLL cells. By comparison, from the results presented here, activation of the alternative NF-κB2/p100 pathway appears not to play a dominant role in promoting BAFF-induced survival of CLL cells (FIG. 13C), which appears to contrast with reported findings in BR3-knockout mice. Our results are closely allied with the finding that B1 cell development is unaffected by disruption of BAFF or BR3, and its development origin differs from that of conventional B2 cells. On the other hand, the viability of CLL cells was markedly suppressed, when we blocked the canonical NF-κB pathway by one of the IKKβ inhibitors (UTC) or through transfection with SR-IκBα(FIGS. 15D, 16B). Therefore, we reasoned that BAFF and APRIL may promote CLL cell survival via the canonical pathway rather than the alternative pathway. Furthermore, activation of the NF-κB canonical pathway may obviate the requirement for activation of the alternative pathway in CLL to promote leukemia cell survival, at least under the in vitro culture conditions used in this study.

A number of selective IKKβ inhibitors have been developed. Several groups reported that IKKβ inhibitors could induce apoptosis of malignant cells, such as myeloma, lymphoma, and myeloid leukemia cells. We examined the effect of one of the IKKβ inhibitor, 5-(4-fluorophenyl)-2-ureido-thiophene-3 carboxylic acid amide (UTC), on CLL cells and purified normal B cells from healthy donors. This compound (FIG. 15A) is identical to TPCA-1 which was reported to be a specific inhibitor of IKK-2 (IKKβ) by Podolin and colleagues. They examined the activity of TPCA-1 against thirteen kinases; IKK-1 (IKKα), IKK-2 (IKKβ), p38α, p38β, p38γ, p38δ, MAPKAPK2, MKK1, MAPK2, COX-1, COX-2, JNK1, and JNK3. The activity of TPCA-1 was 22- and 200 fold selective for IKK-2 versus IKK-1 and JNK3, respectively, and more than 550-fold selective for IKK-2 versus the other ten kinases. Consequently, the compound seems to have high specificity for IKK-2 (IKKβ).

We have shown that UTC, which can block only the canonical pathway, can completely impair the effect of exogenous rhBAFF in CLL cells. Furthermore, the viability of CLL cells cultured with UTC was less than that cultured with medium alone (FIG. 15D). However, UTC did not show any effect on the survival of normal B cells cultured with medium alone (FIG. 15E). These results suggest that the canonical pathway is constitutively activated in CLL cells, even when they are cultured with medium alone. These findings are congruent with those reported by Furman et al. The constitutive activation of the canonical pathway in CLL cells may arise from an autocrine mechanism of BAFF and APRIL, as reported previously. In addition, the survival benefit provided by exogenous rhBAFF to the leukemic cells was negated by co-existence of UTC (FIG. 15D). In contrast to CLL B cells, rhBAFF still had some anti-apoptotic effect on normal B cell even when they were cultured with UTC (FIG. 15E). These data imply that the anti-apoptotic effect of BAFF is highly dependent on the canonical NF-κB pathway in CLL cells, and the manner seems to be something different from normal B cells. In the light of these findings, IKKβ seems to be a potential target to treat patients with this disease. However, we have to consider that inhibition of IKKβ could cause some adverse effects, especially by inhibiting innate and acquired immunity. The short-term use of these inhibitors in cancer patients might be achieved with manageable effects on immune function.

In conclusion, BAFF and APRIL protect CLL B cells from apoptosis. The anti-apoptotic effects of these factors are mediated via activation of the canonical NF-κB pathway. We speculate that inhibitors of IKKβthat inhibit the canonical NF-κB pathway may have therapeutic activity in this disease.

### EXAMPLES

Aspects of the present teachings may be further understood in light of the following examples, which should not be construed as limiting the scope of the present teachings in any way.

### Example 1

### NLC Analysis

Cell Preparation

After obtaining informed consent, blood samples were collected from patients at the University of California, San Diego (UCSD) Medical Center who satisfied diagnostic and immunophenotypic criteria for common B-cell CLL. Blood mononuclear cells were isolated via density-gradient centrifugation with Ficoll-Hypaque (Pharmacia, Uppsala, Sweden). Cells were suspended in fetal calf serum (FCS) containing 5% dimethyl sulfoxide for storage in liquid nitrogen. The viability of the CLL cells was at least 85% at the initiation of cell culture, as assessed by their capacity to exclude propidium iodide (PI, Molecular Probes, Eugene, Oreg.). All CLL mononuclear cell samples contained >95% CD19+/CD5+/CD3− CLL B cells, as assessed by flow cytometry using fluorochrome-conjugated monoclonal antibodies (mAbs) specific for CD19, CD5, or CD3 (BD PharMingen, La Jolla, Calif.). CLL cells were cultured in RPMI-640 (Gibco BRL, Rockville, Md.) supplemented with 10% FCS and penicillin-streptomycin-glutamine (culture medium) in 5%/, CO2 in air at 37° C.

CD14+ blood mononuclear cells or CD19+B cells of healthy donors were isolated from the buffy-coat of blood samples collected from adult volunteers at the San Diego Blood Bank (San Diego, Calif.), as described. CD14+ cells were cultured with isolated CLL B cells in culture medium at cell-densities of 1×105/ml and 1×107/ml, respectively. After 10 to 14 days, the plates were rinsed free of the nonadherent CLL cells. The adherent NLC were then removed for analyses, as described.

Reagents

Anti-human BAFF mAb was purchased from RDI (Flanders, N.J.). Isotype control mouse IgG1 (MOPC-21) and fluorescein isothiocyanate (FITC)-conjugated anti-mouse IgG1 was purchased from BD PharMingen. Phycoerythrin (PE)-conjugated anti-human BAFF mAb was purchased from R&D Systems (Minneapolis, Minn.). Goat-anti-human APRIL (R15) polyclonal antibody was from Santa-Cruz Biotechnology (Santa Cruz, Calif.). FITC-conjugated ant-goat IgG was from Rockland (Gilbertsville, Pa.). Recombinant human BAFF (rhBAFF) was a kind gift from Dr. G Zhang (National Jewish Medical and Research Center, Colorado). Recombinant human APRIL MegaLigand and BCMA-Fc were purchased from Alexis Biochemicals (San Diego, Calif.). BAFF-R:Fc and Control Ig were purchased from R&D Systems (Minneapolis, Minn.). We received the CXCR4 antagonist 4F-benzoyl-TE14011 (4F), which specifically can inhibit the activity of SDF-1α, as a gift from Dr. N. Fujii (Graduate School of Pharmaceutical Sciences, Kyoto University, Japan).

Cell Isolation

Isolated blood mononuclear cells of patients with CLL were incubated with saturating amounts of “Dynabeads” coated with anti-CD2 or anti-CD14 mAbs (Dynal A.S. Oslo, Norway). Bead-bound cells were removed with a strong magnetic field. Following depletion, less than 0.5% of cells were CD2+ or CD14+, respectively, whereas more than 99% were CD19+, as assessed via flow cytometry (data not shown). Peripheral normal CD19+B cells were purified from the buffy-coat of blood samples collected from adult volunteers at the San Diego Blood Bank using CD19-Dynabeads and Detach A Bead (Dynal), following manufacturer's instruction. The purity of the isolated B cells was >95%, as assessed by flow cytometry using a fluorochrome-conjugated anti-CD19 mAb that does not compete with the anti-CD19 mAb used for prior positive selection.

Real-Time Quantitative RT-PCR

Total RNA was isolated from normal CD14+ cells, NLC, normal peripheral B cells, and CLL cells before or after depletion of CD14+ cells, using RNeasy Mini Kit (QIAGEN, Valencia, Calif.). In other experiments, CD14+ monocytes were added to isolated CLL B cells at the indicated ratio and total RNA was made from each sample. To remove contaminating DNA, the isolated RNA was treated with RQ1 RNase-Free DNase (Promega, Madison, Wis.) according to the manufacturer's instructions. First-strand cDNA synthesis was performed with SuperScript™ First-Strand Synthesis System for RT-PCR (Invitrogen, Carlsbad, Calif.). For Real-time PCR, SYBR Green PCR Master Mix (Applied Biosystems, Foster city, CA) was used with 300 nmol/l forward and reverse primers in a final volume 50 μl for each reaction. Amplification primers were as follows: human BAFF 5′ ACCGCGGGACTGAAAATCT 3′ and 5′ CACGCTTATITCTGCTGTTCTGA 3′, human APRIL 5′-CTGCACCTGGTTCCCATTAAC-3′ and 5′-AAGAGCTGGTTGCCACATCA-3′, human glyceraldehyde-3-phosphate dehydrogenase (GAPDH) 5′ ACGGATTTGGTCGTATTGGGC 3′ and 5′ TTGACGGTGCCATGGAATTTG 3′. Each sample was run in duplicate. The polymerase chain reactions were performed using GeneAmp 5700 Detection System (Applied Biosystems) with an initial incubation at 95° C. for 10 minutes, followed by 40 cycles, each cycle consisting of a one minute incubation at 60° C., followed by a fifteen second denaturation step at 95° C. For each run, serially diluted cDNA of U937 cells were used in samples run in parallel to standardize the assay. We determined the cell equivalence (CE) numbers of BAFF, APRIL, and GAPDH mRNA in each sample using the 7700 sequence detector (Applied Biosystems), using the standard curve generated from the diluted U937 cells. The unit number showing relative BAFF or APRIL mRNA level in each sample was determined as a value of BAFF or APRIL CE normalized with GAPDH CE. Melting curve analysis was performed to assess the specificity of PCR product. Following 40 cycles of PCR, samples were heated to 95° C. for 30 seconds, and 60° C. for 20 seconds, then heated to 95° C. at a ramp rate of 0.2° C./second. Melting curves for each sample were drawn with 5700 sequence detector software (Applied Biosystems).

Flow Cytometry

The cells were stained with saturating amounts of antibodies for 30 minutes at 4° C. in Deficient RPMI-1640 supplemented with 0.5% bovine serum albumin (FACS buffer), washed 2 times, and then analyzed on a FACSCalibur (Becton Dickinson, Mountain View, Calif.). Flow cytometry data were analyzed using FlowJo software (Tree Star, San Carlos, Calif.).

Immunofluorescence Staining

CD14+ monocytes were cultured with CLL B cells on Lab-Tek chambered cover glass (Nalge Nunc International, Naperville, Ill.) for immunofluorescence staining, as described.10 After 14 days, the cells were prepared for immunofluorescence staining using the Cytofix/Cytoperm Kit (BD PharMingen), as per the manufacturer's instructions. The fixed and permeabilized cells were incubated with control antibodies, PE-conjugated anti-BAFF mAb and FITC-anti-CD19 (BD PharMingen), or goat-anti-APRIL IgG and PE-anti-CD19 (BD PharMingen). The latter was counterstained with FITC-conjugated anti-goat IgG to detect cell-bound goat antibody. Hoechst 33342 (Molecular Probes, Eugene, Oreg.) was used to stain the nuclei. Optical sections of fluorochrome-labeled cells were captured with a Delta-Vision deconvolution microscope system (Applied Precision, Issaquah, Wash.) of the Digital Imaging Core of the UCSD Cancer Center.

Immunoblot Analysis

Cell lysates were prepared with RIPA buffer (10 mM Tris (pH 7.4), 150 mM NaCl, 1% Triton x 100, 1% deoxycholate, 0.1% SDS, 5 mM EDTA), containing 1 mM PMSF, 0.28 TIU/ml aprotinin, 50 μg/ml leupeptin, 1 mM benzamidine, 0.7 μg/ml pepstatin. Lysates were normalized for total protein (20 μg), subjected to SDS-PAGE (4-15% gradient gels, Bio-Rad, Hercules, Calif.) and immunoblot assay. We incubated the blots with secondary antibodies that were conjugated with horseradish peroxidase. Blots then were prepared for enhanced chemiluminescence (ECL) detection system (Amersham, Little Chalfont, Buckinghamshire, UK) and subsequent autoradiography with Super RX film (Fuji, Tokyo, Japan). The mouse mAb against APRIL (APRIL8) was from Alexis Biochemicals. The mouse mAb against inhibitor of kappa B-α (IκκBα was from Imgenex (San Diego, Calif.). The antibodies against anti-phosho-MAP kinase Erk1/2 and anti-MAP kinase Erk1/2-CT were purchased from Upstate Biotechnology. Antibodies against AKT or phospho-AKT (Ser473) were from Cell Signaling (Beverly, Mass.). Rabbit polyclonal antibodies (Mcl-1, Bcl-2, and Bax) were raised against synthetic peptides.21 Also primary antibodies included β-actin (Sigma Immunochemicals, St Louis, Mo.). Anti-p52 and anti-p65 antibodies were purchased from Upstate Biotechnology.

Subcellular Fractionation and Detection of Cytoplasmic or Nuclear NF-κB

For fractionation experiments, cells were collected by centrifugation and washed with PBS. The cell pellet containing 5×106 cells was suspended in 100 μl of hypotonic buffer (50 mM Tris (pH7.4), 5 mM EDTA, 10 mM NaCl, 0.05% NP-40, 1 mM PMSF, 10 μg/ml Aprotinin, 10 μg/ml Leupeptin, 10 μg/ml Pepstatin, 10 mM β-Glycerophosphate, 1 mM Na-Vanadate, 1 mM NaF). After 10 minutes the lysate was spun and the supernatant was collected as cytoplasmic lysates. The pellet was washed 5 times in hypotonic buffer containing 0.1% NP-40. The remaining pellet was suspended in 100 μl RIPA buffer containing protease and phosphatase inhibitors. After an appropriate amount of 3× sample buffer (200 mM Tris (pH 6.8), 30 mM EDTA, 30% Glycerol, 6% SDS) was added, the sample was boiled for 10 minutes, spun for 10 minutes and the supernatant was recovered as nucleus lysates. Anti-NF kappa B p52 and p65 were purchased from Upstate Biotechnology. Anti-SP-1 was purchased from Santa Cruz Biotechnology (Santa Cruz, Calif.).

Measurement of Cell Viability

Freshly thawed CLL B cells were cultured at the concentration of 1×106/ml under various conditions. Determination of CLL cell viability in this study was based on the analysis of mitochondrial transmembrane potential (Δψm) using 3,3′-dehexyloxacarbocyamine iodine (DiOC6) and cell membrane permeability to PI, as described.22 For viability assays, 100 μl of the cell culture was collected at the indicated time points and transferred to polypropylene tubes containing 100 μl of 60 nmol/l DiOC6 (Molecular Probes) and 10 μg/ml PI in FACS buffer. The cells then were incubated at 37° C. for 15 minutes and analyzed within 30 minutes by flow cytometry using a FACSCalibur (Becton Dickinson). Fluorescence was recorded at 525 nm (FL-1) for DiOC6 and at 600 nm (FL-3) for PI.

Statistical Analysis

Results are shown as mean±S.D. of at least 3 samples each. For statistical comparison between groups, the Student t test or the Bonferroni t test was used. Analyses were performed using Glanzman's “Primer of Biostatstics” software (McGraw-Hill Inc., New York, N.Y.).

### Example 2

### IKKβ Inhibition

Cell Preparation

After informed consent was obtained per the Declaration of Helsinki, blood samples were collected from patients at the University of California, San Diego (UCSD) Medical Center who satisfied diagnostic and immunophenotypic criteria for common B-cell CLL. Blood mononuclear cells were isolated via density-gradient centrifugation with Ficoll-Hypaque (Pharmacia, Uppsala, Sweden). Cells were suspended in FCS containing 5% DMSO for storage in liquid nitrogen. The viability of the CLL cells was at least 85% at the initiation of cell culture, as assessed by their capacity to exclude propidium iodide (PI) (Molecular Probes, Eugene, Oreg.). All CLL mononuclear cell samples contained more than 95% CD19+/CD5+/CD3−CLL B cells, as assessed by flow cytometry using fluorochrome-conjugated monoclonal antibodies (mAbs) specific for CD19, CD5, or CD3 (BD PharMingen, La Jolla, Calif.). CLL cells were cultured in RPMI-1640 (Gibco, Rockville, Md.) supplemented with 10% FCS and penicillin-streptomycin-glutamine (culture media) in 5% CO2 in air at 37° C.

CD19+ B cells of healthy donors were isolated from the buffy coat of blood samples collected from adult volunteers at the San Diego Blood Bank (San Diego, Calif.), as described.

Cell Isolation

Isolated blood mononuclear cells of patients with CLL were incubated with saturating amounts of Dynabeads coated with anti-CD2 or anti-CD14 mAbs (Dynal A.S. Oslo, Norway). Beadbound cells were removed with a strong magnetic field. Following depletion, less than 0.5% of cells were CD2+ or CD14+, whereas more than 99% were CD19+, as assessed via flow cytometry (data not shown). Peripheral normal CD19+ B cells of healthy donors were purified from the buffy coat of blood samples using CD19 Dynabeads and Detach A Beads (Dynal), following the manufacturer's instructions. The purity of the isolated B cells was more than 95%, as assessed by flow cytometry using a fluorochrome-conjugated anti-CD19 mAb that does not compete with the anti-CD19 mAb used for prior positive selection (data not shown).

Reagents

rhBAFF was a kind gift from Dr. G Zhang (National Jewish Medical and Research Center, Denver, Colo.). rhAPRIL was purchased from Alexis Biochemicals (San Diego, Calif.). We obtained anti-human BR3 antibody, recombinant human BR3-Fc, recombinant human BCMA-Fc, and control human IgG from Genentech (South San Francisco, Calif.) and Biogen Idec (Cambridge, Mass.). Recombinant human TNF-α (rhTNF-α) was purchased from R&D Systems (Minneapolis, Minn.).

Antibodies

Rat anti-BCMA and anti-TACI mAbs were purchased from Alexis Biochemicals. The relevant isotype control mAbs were from BD PharMingen. PE-labeled mouse anti-rat IgG was from Santa Cruz Biotechnology (Santa Cruz, Calif.). Biotinylated anti-BR3 antibody and mouse IgG2a isotype control were obtained from Genentech. Allophycocyanin-labeled streptavidin was purchased from BD PharMingen. The mouse mAb against IκBαwas from Imgenex (San Diego, Calif.). Rabbit anti-phospho-IκBα(Ser32) antibody was from Cell Signaling Technology (Beverly, Mass.). Mouse anti-p52 and rabbit anti-p65 antibodies for immunoblot analysis were from Upstate Biotechnology (Lake Placid, N.Y.). Mouse anti-HA mAb was from Roche diagnostics (Indianapolis, Ind.). Anti-Sp-1 was purchased from Santa Cruz Biotechnology. Also primary antibodies included β-actin (Sigma Immunochemicals, St Louis, Mo.). Those of skill in the art will recognize that the antibodies of the present invention can be of human origin or humanized according to Biological Methods below.

Preparation of IKKβ Inhibitor

We synthesized one of the IKKβ inhibitors, 5-(4-fluorophenyl)-2-ureido-thiophene-3 carboxylic acid amide (UTC). UTC was prepared in three steps according to the procedure described in the PCT patent application WO 02/30353 A2 beginning with 2-(4-fluorophenyl)ethanol. Oxidation of this alcohol to the corresponding aldehyde using pyridinium chlorochromate followed by condensation with 2-cyanoacetamide and sulfur provided the substituted thiophene, 2-amino-5-(4-fluorophenyl)thiophene-3-carboxamide. Finally, the amino function of this thiophene was converted to the ureido group by reaction with trichloroacetylisocyanate followed by treatment with ammonia to yield the final product UTC.

Flow Cytometry

To analyze membrane expression of BCMA, TACI, and BR3, the cells were stained with saturating amounts of primary antibodies for 30 minutes at 4° C. in Deficient RPMI-1640 or PBS supplemented with 0.5% BSA (FACS buffer), washed 2 times, and then counterstained with PE-labeled secondary antibody or allophycocyanin-labeled streptavidin for 30 minutes at 4° C. After washed 2 times, cells were analyzed by FACSCalibur (Becton Dickinson, Mountain View, Calif.). Flow cytometry data were analyzed using FlowJo software (Tree Star, San Carlos, Calif.).

Measurement of Cell Viability

Freshly thawed CLL B cells were cultured at the concentration of 1×106/mL under various conditions. Determination of CLL cell viability in this study was based on the analysis of mitochondrial transmembrane potential (Δψm) using 3,3′-dehexyloxacarbocyamine iodine (DiOC6) and cell membrane permeability to PI, as described. For viability assays, 100 μl of the cell culture was collected at the indicated time points and transferred to polypropylene tubes containing 100 μl of 80 nmol/l DiOC6 (Molecular Probes) and 2 μg/ml PI in FACS buffer. The cells then were incubated at 37° C. for 15 minutes and analyzed within 30 minutes by flow cytometry using a FACSCalibur (Becton Dickinson). Fluorescence was recorded at 525 nm (FL-1) for DiOC6 and at 600 nm (FL-3) for PI.

Immunoblot Analysis

Cell lysates were prepared with radioimmunoprecipitation assay (RIPA) buffer (10 mM Tris [pH 7.4], 150 mM NaCl, 1% Triton X-100, 0.1% deoxycholate, 0.1% SDS, 5 mM EDTA), containing 1× complete protease inhibitor cocktail (Roche diagnostics), 1 mM sodium fluoride (NaF), and 1 mM sodium vanadate (Na3VO4). Lysates were normalized for total protein (25 μg) and subjected to SDS-PAGE (4-15% gradient gels, Bio-Rad, Hercules, Calif.) and immunoblot assay. We incubated the blots with secondary antibodies that were conjugated with horseradish peroxidase. Blots then were prepared for enhanced chemiluminescence (ECL) detection system (Amersham, Little Chalfont, United Kingdom) and subsequent autoradiography with Super RX film (Fuji, Tokyo, Japan).

Subcellular Fractionation and Detection of Cytoplasmic or Nuclear NF-κB

For fractionation experiments, cells were collected by centrifugation and washed with PBS. The cell pellet containing 5×106 cells was suspended in 100 μl of hypotonic buffer (50 mM Tris [pH7.4], 5 mM EDTA, 10 mM NaCl, 0.05% Nonidet P40 [NP-40]), containing 1× complete protease inhibitor cocktail, 1 mM NaF, and 1 mM Na3VO4. After 10 minutes, the lysate was spun and the supernatant was collected as cytoplasmic lysates. The pellet was washed 5 times in hypotonic buffer containing 0.1% NP-40. The remaining pellet was suspended in 100 μl RIPA buffer containing protease and phosphatase inhibitors. After 10 minutes, the lysate was spun for 15 minutes and the supernatant was recovered as nuclear lysates.

Electrophoretic Mobility Shift Assays

Nuclear proteins were extracted using a nuclear extraction kit (Pierce, Rockford, Ill.) in presence of 1× complete protease inhibitor cocktail (Roche diagnostics). Total protein was measured using a modified Bradford test (Bio-Rad, Hercules, Calif.). 2 μg of nuclear protein extracts were incubated on ice for 30 min with antibodies to p50 and p65 (Santa Cruz Biotechnology). Later, a radiolabeled double stranded probe that encompassed the κB1 site was added, followed by incubation at room temperature for 30 min. Samples were loaded on a 6% acrylamide gel and run at 150 volts for three and a half hours.

Plasmid

A pcDNA3-based expression vector for hemagglutinin (HA)-tagged IκBα mutant (S32A/S36A), also referred to as “SR-IκBα”, was kindly provided by M. Karin (UCSD, La Jolla, Calif.). Mutation of SR-IκBα was confirmed by DNA sequencing. pmaxGFP (green fluorescent protein) was obtained from Amaxa (Gaithersburg, Md.).

Cell Transfection

HeLa cells were maintained in DMEM (Gibco) supplemented with 10% FCS. For transfection, cells at 60-80% confluence were transfected with SR-IκBα or empty pcDNA3 vector using Lipofectin Plus (Invitrogen, Carlsbad, Calif.), according to the manufacturer's instructions, and analyzed 24 hours after transfection.

CLL cells were transfected using the Amaxa nucleofection technology (Amaxa). Cells were resuspended in solution from human B cell nucleofector kit, also available as part of Amaxa cell optimization kit, according to the manufacturer's instructions. Briefly, 100 μl of 5×106 cell suspension mixed with 5 μg cDNA was transferred to the provided cuvette and nucleofected with an Amaxa Nucleofector apparatus (Amaxa). Cells were transfected using the U-15 pulsing parameter and immediately transferred into wells containing 37° C. pre-warmed culture medium in 12-well plates. After transfection, cells were cultured from 4 to 48 hours before analyzing by FACS. pmaxGFP was used to gauge transfection efficiency.

Statistical Analysis

Results are shown as mean±S.D. of at least 5 samples each. For statistical comparison between groups, the Bonferroni t test or the Student paired t test was used. Analyses were performed using PRISM software version 3.0 (GraphPad Software, San Diego, Calif.).

Biological Methods

Methods involving conventional molecular biology techniques are generally known in the art and are described in detail in methodology treatises such as molecular cloning: a laboratory manual, 2nd ed., vol. 1-3, ed. Sambrook et al., Cold Spring Harbor Laboratory Press, Cold Spring Harbor, N.Y., 1989; and current protocols in molecular biology, ed. Ausubel et al., Greene Publishing and Wiley-Interscience, New York, 1992 (with periodic updates). Various techniques using polymerase chain reaction (PCR) are described, e.g., in Innis et al., pcr protocols: a guide to methods and applications, Academic Press: San Diego, 1990. PCR-primer pairs can be derived from known sequences by known techniques such as using computer programs intended for that purpose. The Reverse Transcriptase Polymerase Chain Reaction (RT-PCR) method used to identify and amplify certain polynucleotide sequences within the invention may be performed as described in Elek et al., In vivo, 14:172-182, 2000). Methods and apparatus for chemical synthesis of nucleic acids are provided n several commercial embodiments, e.g., those provided by Applied Biosystems, Foster City, Calif., and Sigma-Genosys, The Woodlands, Texas. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., gene therapy: principles and applications, ed. T. Blackenstein, Springer Verlag, 1999; gene therapy protocols (methods in molecular medicine), ed. P. D. Robbins, Humana Press, 1997; and retro-vectors for human gene therapy, ed. C. P. Hodgson, Springer Verlag, 1996.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended 1D fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individual indicated to be incorporated by reference in its entirety for all purposes. Citation of a reference herein shall not be construed as an admission that such is prior art to the present invention. Specifically intended to be within the scope of the present invention, and incorporated herein by reference in its entirety, is the following publication: Nurselike cells express BAFF and APRIL, which can promote survival of chronic lymphocytic leukemia cells via a paracrine pathway distinct from that of SDF-1 alpha. Blood, 2005 Aug. 1; 106(3):1012-20.

What is claimed is:

1. A method for regulating apoptosis in a cell, the method comprising:
contacting the cell with an agent capable of neutralizing BAFF or APRIL, such that an activity of BAFF or APRIL is inhibited.

2. A method according to claim 1, wherein the agent is a soluble form of BCMA.

3. A method according to claim 2, wherein the soluble form of BCMA is BCMA-Fc.

4. A method according to claim 2, wherein the agent is a soluble form of TACI.

5. A method according to claim 4, wherein the soluble form of TACI is TAC-Fc.

6. A method according to claim 2, wherein the agent is UTC.

7. A method according to claim 1, wherein the agent is selected from the group consisting of a small molecule, protein, peptide, peptidomimetic, nucleic acid molecule or any combination thereof.

8. A method according to claim 7, wherein the polypeptide is an antibody.

9. A method according to claim 7, wherein the polypeptide is a soluble BCMA receptor.

10. A method according to claim 9, wherein the soluble BCMA receptor is BCMA-Fc.

11. A method according to claim 7, wherein the receptor is a soluble TACI receptor.

12. A method according to claim 11, wherein the soluble TACI receptor is TAC-Fc.

13. A method according to claim 1, wherein the cell is a neoplastic cell.

14. A method for treating leukemia in a subject, the method comprising:
contacting a subject with an agent capable of neutralizing BAFF or APRIL, such that an activity of BAFF or APRIL is inhibited.

15. A method according to claim 14, wherein the agent is a polypeptide.

16. A method according to claim 14, wherein the agent is an antibody.

17. A method according to claim 14, wherein the agent is a BCMA receptor.

18. A method according to claim 17, wherein the soluble BCMA is BCMA-Fc.

19. A method according to claim 14, wherein the agent is a soluble TACI receptor.

20. A method according to claim 19, wherein the soluble TACI receptor is TAC-Fc.

21. A method according to claim 14, wherein the agent is UTC.

22. A method for identifying a candidate CLL inhibiting compound, the method comprising:
a) contacting a test compound with a CLL cell and one of BAFF or APRIL; and
b) detecting the level of apoptosis in the presence of said test compound and one of BAFF or APRIL as compared to the level of apoptosis in the absence of said test compound, wherein a decreased level of apoptosis in the presence of said test compound indicates that the test compound is a CLL inhibiting compound.
